(19) United States
(12) Patent Application Publication
(10) Pub. No.: US 2008/0177166 A1
(43) Pub. Date: Jul. 24, 2008

(54) Ultrasensitive amperometric saliva glucose sensor strip

(75) Inventors: Allan D. Pronovost, San Diego, CA (US); Michael Hickey, Laguna Hills, CA (US)

Correspondence Address: FOLEY & LARDNER LLP, 111 HUNTINGTON AVENUE, 26TH FLOOR, BOSTON, MA, 02199-7610, US

(73) Assignee: PROVEX TECHNOLOGIES, LLC

(21) Appl. No.: 11/715,719

(22) Filed: Mar. 7, 2007

Related U.S. Application Data
(60) Provisional application No. 60/881,126, filed on Jan. 18, 2007.

(51) Int. Cl. A61B 5/00

(52) U.S. Cl. 600/365

(57) ABSTRACT

Methods and apparatus for measuring a carbohydrate in a fluid are presented. An amperometric glucose sensor system suitable for glucose monitoring in a biological sample other than blood includes a support member having a sample region defined thereon in fluid communication with a measurement zone including an electrode having an exposed surface area. Biological sample fluid is transported from the sample region to the measurement zone, which includes an exposed catalyst in communication with the electrode providing a minimum sensitivity of at least about 50 micromolar glucose concentration and a noise level of less than about 0.5 nA/μM/mm². In some embodiments, at least one of a nanofiltration material and a porous absorbent material is provided between the sample region and the measurement zone.

### RELATED APPLICATIONS

This application claims the benefit of U.S. Provisional Application No. 60/881,126, filed Jan. 18, 2007. The entire teachings of the above application are incorporated herein by reference.

### FIELD OF THE INVENTION

The present invention relates to the measurement of carbohydrate in a fluid and uses thereof. Specifically, the invention is directed to the field of glucose measurement in the salivary fluid of a subject. The invention provides an electrochemical sensor and method for the amperometric measurement of salivary glucose in a subject.

### BACKGROUND OF THE INVENTION

Diabetes is a disease in which the body does not produce or properly use insulin. Insulin is a hormone that is needed to convert sugar, starches and other food into energy needed for daily life. Diabetes is a major health concern as it is estimated that there are more than 18 million people in the United States who have diabetes. The cause of diabetes is not completely understood, although both genetics and environmental factors such as obesity and lack of exercise appear to play roles. Routine glucose monitoring plays a central role in diabetes management.

Glucose sensors are in routine use for blood glucose monitoring. Blood glucose monitors are meter devices that are electrochemically based and read differences in current or read color changes produced on specially treated reagent strips by glucose concentrations in the patient's blood. Blood glucose monitors measure blood glucose concentrations using a reagent strip, cartridge or cuvette and a drop of blood from a finger puncture. Used at home, blood glucose monitors allow people with diabetes to detect and treat fluctuation in blood glucose levels. The normal fasting blood glucose concentrations ranges from about 70 to about 110 mg/dL in blood serum or plasma, although capillary blood glucose concentrations can be higher by 10-15%. A person with diabetes can adjust insulin, dosage of oral medications food intake, or exercise in response to the monitor's readings to achieve normoglycemia. The Diabetes Control and Complications Trial conducted from 1983 to 1993 by the National Institute of Diabetes and Digestive and Kidney Diseases (NIDDK), showed that maintaining normoglycemia facilitates treatment designed to reduce the incidence and severity of diabetes-related eye, kidney, and nerve diseases.

Some portable blood glucose monitors use reflectance photometry to measure the amount of light produced by a light-emitting diode (LED) and reflected from a reagent-impregnated test pad that has reacted with a drop of blood. Some units use absorbance photometry, an optical reading method that measures glucose concentration using two wavelengths, rather than the single wavelength used by reflectance photometry. Other blood glucose monitors use electrochemical methodology where electrodes measure the current that is produced by the conversion of glucose to gluconic acid via glucose oxidase, glucose dehydrogenase or hexokinase when blood is applied to the test strip. As noted above, the most commonly used laboratory diagnostic procedures involve the analyses of the cellular and chemical constituents of blood. (Kaufman and Lamster, Crit. Rev. Oral Biol. Med., 13(2):197-212, 2002).

All blood glucose meters are designed to work specifically with blood and are designed and tailored to meet the physiological requirements for measuring glucose in blood. They are not designed to work with other body fluids nor can they very effectively do so, if at all. As one example, blood samples are notoriously oxygen deprived. As such enzyme mediators and cofactors are used as part of the complex enzyme chemistry of the sensor strip as the means to extend the dose response curve for signal to match the wide dynamic range of glucose concentrations found in the blood sample. Without such mediator enhancement of enzyme function and signal generation, the dynamic range for blood measurement would not be met and the sensor would not work effectively for the oxygen deprived sample.

### SUMMARY OF THE INVENTION

In one aspect, the invention relates to an apparatus for detecting a concentration of glucose in a biological sample other than blood, such as saliva. The apparatus includes a support member and an electrode disposed thereon having an exposed surface area. The apparatus also includes a sample region having a sample port upon which the biological sample is applied. A lumen is provided having a proximal end in fluid communication with the sample region and a distal end in fluid communication with a measurement zone. The measurement zone has an enzymatic catalyst and provides a minimum sensitivity of at least about 50 micromolar glucose concentration and a noise level of less than about 0.5 nA/μM/mm².

In another aspect, the invention relates to an apparatus for processing a mammalian saliva sample. The apparatus includes a saliva sample port for receiving the saliva sample and a filter in fluid communication with the sample port. The filter includes a nanofiltration material configured to remove high molecular weight contaminants from the saliva sample and a porous absorbent material configured to absorb at least a portion of the saliva sample. The nanofiltration material and the absorbent material are configured to filter the saliva sample.

In yet another aspect, the invention relates to a process for determining glucose levels in a mammalian saliva sample. The saliva sample is received at a sample port. At least a portion of the received saliva sample is transported from the sample port to a measurement zone. The transported saliva sample is combined with an enzymatic catalyst within the measurement zone. A glucose level of the saliva sample is measured with a minimum sensitivity of at least about 50 micromolar glucose concentration and a noise level of less than about 0.5 nA/μM/mm².

### BRIEF DESCRIPTION OF THE DRAWINGS

Referring now to the drawings, wherein like reference numerals and letters indicate corresponding structure throughout the several views:

FIG. 1 is a schematic drawing illustrating a top view of an embodiment of the glucose sensor system of the invention.

FIG. 2 is a schematic drawing illustrating a top view of an embodiment of the glucose sensor system of the invention.

FIG. 3A through FIG. 3J are schematic drawings showing the construction of an embodiment of the glucose sensor system of the invention. FIG. 3A shows a top view of a platinum/titanium film useful in the construction of the glucose sensor system of the invention. FIG. 3B shows a top view of the platinum/titanium film of FIG. 3B after laser ablation. FIG. 3C shows a top view of the film of FIG. 3B after applying a dialectric layer. FIG. 3D shows a top view of the film of FIG. 3C after printing of silver/silver chloride. FIG. 3E shows a top view of the film of FIG. 3D after printing of carbon. FIG. 3E shows a top view of a film of FIG. 3D after applying a spacer layer. FIG. 3G shows a top view of the film of FIG. 3F after application of a reagent. FIG. 3H shows a top view of the film of FIG. 3H after application of a lid. FIG. 3I shows a top view of the film of FIG. 3H after application of a nanofilter. FIG. 3J shows a top view of the film of FIG. 3H after application of a molecular filter to complete the glucose sensor system.

FIG. 4 is a schematic drawing illustrating a top perspective view of an embodiment of the glucose sensor system of the invention.

FIG. 5 is a schematic drawing illustrating an exploded top perspective view of the elements to construct an embodiment of the glucose sensor system of the invention.

FIG. 6 is a graph comparing electrical current in micro amps (μA) as a function of glucose concentration (mg/dL) observed with the YSU 2700 glucose sensor and the EZ SMART glucose sensor.

FIG. 7 is a graph comparing the electrical current density (μA/cm²) as a function of hydrogen peroxide concentration (μM) measured by select electrodes of the invention.

FIG. 8 is a graph showing a comparison of response to hydrogen peroxide in artificial saliva for sputtered platinum and platinized carbon parts with lids.

FIG. 9 is a graph showing a comparison the electrical current (μA) as a function of glucose oxidase concentration (wt %) observed at varying glucose concentration (0-5000 μM) versus time.

FIG. 10 is a graph showing a comparison of the effect of varying electrode area on the average current (μA) as a function of hydrogen peroxide concentration (μM).

### DETAILED DESCRIPTION OF THE INVENTION

The problems of oxygen deprivation and a broad dynamic range associated blood samples used with blood glucose meters are not issues found with salivary fluid samples. Salivary fluid samples are oxygen rich, having a dynamic range 50-100 lower than the minimal concentration of glucose found in blood.

Other biologic fluids can theoretically be utilized for the diagnosis of disease, and salivary fluid offers some distinctive advantages. Saliva offers an alternative to serum as a biologic fluid that can be analyzed for diagnostic purposes. Whole saliva contains locally produced as well as serum-derived markers that have been found to be useful in the diagnosis of a variety of systemic disorders. The saliva present in oral salivary glands such as the parotid has been shown to contain low molecular weight substances found in blood. Substances in blood, including analytes of medical interest such as glucose are known to readily permeate the blood vascular membrane border from the blood capillaries, infiltrating the saliva gland. Transit takes on average 20 minutes and analyte levels reflect blood levels albeit lower in concentration.

Salivary fluid can be collected in a non-invasive manner by individuals with modest training, including consumers. No special equipment is needed for collection of the fluid. Diagnosis of disease via the analysis of saliva is potentially valuable for children and older adults, since collection of the fluid is associated with fewer compliance problems as compared with the collection of blood. Fear of blood collection is the major factor for non compliance. Further, analysis of saliva may provide a cost-effective approach for the screening of large populations. (Bailey et al., Pediatr. Clin. North Am., 44:15-26, 1997). There remains a need for improved means of measuring salivary glucose.

Definitions and Abbreviations.

As used herein, the following definitions define the stated term:

The term “amperometry” as used herein includes steady-state amperometry, chronoamperometry, and Cottrell-type measurements.

A “biological sample” is any body fluid in which the analyte can be measured, for example, blood (which includes whole blood and its cell-free components, such as, plasma and serum), interstitial fluid, dermal fluid, sweat, tears, urine and saliva.

A “counter electrode” refers to one or more electrodes paired with the working electrode, through which passes an electrochemical current equal in magnitude and opposite in sign to the current passed through the working electrode. The term “counter electrode” is meant to include counter electrodes which also function as reference electrodes (i.e., a counter/reference electrode) unless the description provides that a “counter electrode” excludes a reference or counter/reference electrode.

An “electrochemical sensor” is a device configured to detect the presence of and/or measure the concentration of an analyte via electrochemical oxidation and reduction reactions. These reactions are transduced to an electrical signal that can be correlated to an amount or concentration of analyte.

A “fill detect electrode” is an electrode that detects partial or complete filling of a sample chamber and/or measurement zone with sample.

The “measurement zone” is defined herein as a region of the sample chamber sized to contain only that portion of the sample that is to be interrogated during an analyte assay.

A “layer” is one or more layers.

A “redox mediator” is an electron transfer agent for carrying electrons between the analyte and the working electrode, either directly or through another electron transfer agent.

A “reference electrode” includes a reference electrode that also functions as a counter electrode (i.e., a counter/reference electrode) unless the description provides that a “reference electrode” excludes a counter/reference electrode.

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

A “working electrode” is an electrode at which analyte is electrooxidized or electroreduced with or without the agency of a redox mediator.

Blood Glucose Monitor Technology is Not Useful to Detect Salivary Glucose

Blood glucose monitors are too insensitive to detect salivary glucose. On average, the lower limit of detection (LOD) of fingerstick-type blood glucose monitors commercially available exceeds 40 mg/dL. Saliva glucose levels range from about 0.0 to about 5 mg/dL whereas the dynamic range for blood glucose is about 40 to about 500 mg/dL. That is, saliva glucose levels are about 1/50^{th} of the level that is found in blood. Accordingly, fingerstick-type blood glucose sensors are not sensitive enough to measure the low levels of glucose in whole saliva or stimulated saliva or stimulated processed saliva.

Although the construction of blood glucose sensors is adequate for the high levels of glucose found in blood, there are inherent design features, materials, and enzyme chemistry choices that readily prohibit their use for saliva. As such, simple “fine tuning” of blood glucose sensors for use with saliva is not feasible as there are many inherent limitations in blood glucose sensors. These limitations include, but are not limited to, e.g., the solid-phase plastic supports used for the electrodes, actual electrode composition, inadequate electrode conductive efficiency, and inefficient enzyme turnover. The combination of these factors results in high noise, low signal and lack of sensitivity. Blood sensors have evolved for use primarily with blood to address the physiological requirements for measurement of glucose in an oxygen depleted sample rendering them ineffective for use with other body fluids like saliva which have different requirements from a physiological, chemical, interference factor, and assay standpoint.

The inherent high noise of blood glucose monitor devices is generally attributable to the plastic materials used as solid-phase supports for the electrodes. Electrodes are cast by a variety of methods onto plastic supports. The composition of the plastic, the method used for depositing the electrodes, the electrode configuration, the electrode materials, the enzyme chemistry used and other design factors all contribute to the noise of the system. Measurement of saliva glucose requires a sensitivity in the range of about 0 to about 5.0 mg/dL glucose (which is equivalent to about 0 to about 500 μM H₂O₂ using glucose oxidase). The background noise contribution for this level of sensitivity needs to be at or near zero as there is no room for correction or subtraction of it.

The choice of electrode material is critical. Typically working electrodes for blood glucose sensors are carbon based which are adequate for blood use. Carbon electrodes lack the conductivity necessary, however, to measure glucose in saliva electrochemically by amperometric means. Saliva glucose produced current levels in the low nanoampere range as determined using the solid platinum electrodes of the YSI 2700 laboratory based glucose analyzer, commercially available from YSI Incorporated of Yellow Springs, Ohio. The concentration level of salivary glucose is some 50 to 100 fold below the lower detection limit of conventional carbon electrode chemistry as used for blood monitoring.

The electrodes used and the sensitivity required generally dictates the enzyme chemistry that can be employed. Most electrochemical based blood glucose sensors employ a combination of enzymes to allow electron generation throughout the broad dynamic range (e.g., about 40 to about 500 mg/dL) required for blood glucose in a fingerstick-type whole blood sample. The broad dynamic range necessitates a dual enzyme channeling system wherein, e.g., glucose oxidase and horseradish peroxidase are coupled to assure turnover and supply of substrates for enzyme catalysis. Low oxygen levels in blood would prohibit the use of glucose oxidase alone as enzyme wherein β-D-glucose couples with molecular oxygen in the presence of glucose oxidase enzyme and is catalyzed to produce D-glucono-1,5-lactone plus H₂O₂. The H₂O₂ generated can be readily measured directly on a suitable electrode with high sensitivity. Blood cannot employ this enzyme chemistry due to oxygen depletion in the viscous sample.

The Amperometric Glucose Sensor System of the Invention.

The present invention relates to the measurement of carbohydrate in a fluid and uses thereof. Specifically, the invention is directed to the field of glucose measurement in the saliva of a subject. This invention provides an electrochemical glucose sensor strip system suitable for salivary glucose monitoring (i.e., glucose sensor system). In one embodiment of the salivary glucose sensor system of the invention the means of detecting glucose is by amperometry, a process for determining the concentration of a material in a sample solution by measuring electric current passing through a cell containing the solution. That is, the invention provides an amperometric glucose sensor system. The sensor strip utilizes a platinum electrode film for the detection of H₂O₂ generated from the breakdown of glucose by glucose oxidase in a biological sample. As noted above, the term “biological sample” is intended to include, but is not limited to, e.g., tissues, cells and biological fluids isolated from a subject, as well as tissues, cells and fluids present within a subject. In one embodiment, the biological sample is saliva. The saliva may be stimulated, processed saliva.

Saliva contains a variety of components that can actively interfere with salivary glucose monitoring following collection of either non-stimulated or stimulated saliva after appropriate fasting. The probability and level of such interference is enhanced over time the longer the glucose is exposed to interfering components. Saliva is a viscous, dense, sticky fluid innately containing microorganisms like bacteria and fungi, intact human cells, cellular debris, and many soluble materials. Some of the factors that can effect glucose detection and monitoring in saliva include: the enzymatic degradation of glucose (by enzymes normally found in the mouth); degradation of glucose by microbes wherein glucose is a food source; host cellular metabolism for energy; adherence of glucose to mucins, polysaccharides, and proteinaceous materials; and the inherent molecular instability of the glucose molecule itself over time owing to isomerization and other intramolecular variations (glucose exists in a left and right form, the ratio of which can vary spontaneously; glucose also converts depending upon pH and ionic strength to other isomeric forms such as fucose and mannose; glucose also changes structural form based on rotation around anomeric carbon 2).

The glucose sensor system of the present invention is useful in a method for salivary processing and glucose detection wherein there is immediate, almost instantaneous, and efficient (i) active processing and delivery of glucose in stimulated whole saliva away from the interfering substances to the detection means, for actively, not passively, removing glucose away from the interfering substances described above; (ii) this is followed by detection of glucose within the sample liquid upon delivery to the sensor means without the need for further sample elution or processing; and (iii) subsequent quantitation by an electrochemical sensor system with sufficient sensitivity and resolution to measure the lower levels of glucose found in salivary fluid.

Ultrasensitive measurement of salivary glucose can be established for a subject by using a system including a platinized electrode deposited on a plastic film (e.g., polyvinylidene difluoride (PVDP) or polyethylene terephthalate (PET)) as solid phase. The use of platinized electrodes deposited on a plastic film has the advantage of a high detection signal with low noise. That is, the glucose sensor system of the invention exhibits essentially no noise as compared to blood sensors. Furthermore, since the stimulated saliva processing methods of the invention result in high oxygenation of the samples upon processing, it is possible to use H₂O₂ directly for glucose detection as this can be measured directly on the platinum electrode film. That is, the glucose sensor system of the invention is designed to work with oxygen rich samples like stimulated saliva and does not work with oxygen deprived samples like whole blood. This facilitates, in turn, the use of glucose oxidase alone as the enzyme for generation of H₂O₂ in the saliva glucose sensor. In addition, the nature of the sample and the electrode construction of the glucose sensor system of the invention requires a bare minimum reagent formulation that is stable and requires no mediators (e.g., redox mediator) nor dual enzymes or cofactors. That is, the glucose sensor system of the invention utilizes simple enzyme chemistry with minimal stabilizers (and is however more stable than complex blood sensors with a longer shelf life), and lacks the use of enzyme mediators and other REDOX materials. This sensor design affords very low noise, very high signal and the ultrasensitivity required for the salivary sample type.

The glucose sensor system of the invention is too sensitive for use in blood as it does not perform suitable at glucose levels above about 35 mg/dL. That is, the glucose sensor system of the invention is ultrasensitive (e.g., about 0 to about 30 mg glucose/dl) vs. blood (from about 40 to about 500 mg glucose/dl, or greater), and hence is ineffective for blood use just as blood sensors are ineffective for saliva use. The surface area of the sensor is large and much larger than blood sensors to afford saliva sensitivity and high signal production with small saliva volume requirement. Although the overall length and width of the saliva sensor for convenience of consumer use, is the same as the blood sensor, the internal design is such that the “sensing’ portion in contact with the oxygen rich sample is larger than that designed for the blood sensor. This design allows for several unique features specific to salivary fluid use: use of a small volume of saliva (namely the same as blood), yet affording the sensitivity (e.g., 50 times higher than blood); with much higher signal strength than blood (much higher output electrochemically than blood at about 1/50^{th} the glucose concentration); and with no system noise to impede the signal/noise ratio and analytical threshold sensitivity.

As such, combinations of processes are integrated into the glucose sensor system of the invention to provide a one-step device. Suitable media include any material of appropriate construction for the glucose sensor system required. Media can be membranes, molded material, extruded material, or the like including housing design. Any shape necessary to complete the function can be utilized.

Media can be held together to create the device by any means necessary based on the design. This may include, e.g., compression fit, welding, adhesion, ultrasonic welding, heating, stapling, use of adhesives, etc. Media may also be held together using plastic devices. Plastic devices are well known in the art and can be blow molded, thermoform molded, or extruded molded plastic parts. Any number of plastics and resins can be used with the provision that glucose not bind non-specifically. Alternatively the processing and collection device can be constructed from non-plastic, paperboard materials.

The sensor design is particularly suited for the measurement of glucose in saliva, as the materials used for enzyme catalysis are exhausted relatively quickly. As such, in one embodiment, the glucose sensor system of the invention is a disposable system. The disposable feature of the salivary glucose sensor system of the present invention is unique feature among glucose sensor systems known in the art to date. That is, the glucose sensor system of the invention has a saliva processing layer(s) for removal of debris from saliva samples which is an improvement on methods of salivary glucose testing which relies on a separate saliva collection device. In one embodiment, the saliva processing and flow of sample to the measurement zone of the device is via a passive process (i.e., gravity feed and/or capillary action). Furthermore, the sample volume delivered by processing of saliva in the glucose sensor system of the invention is at least about 7 microliters, sample volume obtained is not limiting for the measurement of glucose in saliva, as it would be with other body fluids. For example, the ability to generate a large volume of saliva fluid allows the sensor technology to be used for saliva but prohibits its use for interstitial fluid as a volume greater than 0.5 microliters cannot be delivered. In the case of interstitial fluid the sensitivity would have to be much higher (e.g., about 20 to about 50 times) to warrant the detection of the overall lower mass of glucose available in the lower volume of fluid delivered. As such the use of platinized electrode films on PVDP or PET in the glucose sensor system of the present invention is well suited for saliva use from an oxygenation, sample volume, and analyte concentration standpoint and affords the means to measure glucose in stimulated processed saliva for saliva glucose monitoring.

In some embodiments, the salivary glucose sensor systems of the invention is fabricated at least in part by screen printing polymer thick film inks on a plastic substrate. In some embodiments of the glucose sensor system of the invention, the plastic substrate is polyester or polycarbonate plastic derivatives, e.g., but not limited to, PET and PVDP. The glucose sensor system of the invention includes one or more electrodes selected from a working electrode, a counter electrode, and fill detect electrode. In a preferred embodiment of the invention the glucose sensor system comprises a working electrode, a counter electrode, and fill detect electrode.

The materials for the printed electrodes may vary. In one embodiment of the glucose sensor system, the fill detect and counter electrodes, as well as the leads and contact pads, are printed using conductive ink, Conductive ink compositions useful the glucose sensor system of the invention include, but are not limited to a silver, carbon, or blended conductive ink. Several inks are useful to print the working electrode of the glucose, including, e.g., but not limited to, carbon, platinum, carbon/platinum, or other conductive material suitable for the detection of peroxide in the sample.

In some embodiments of the glucose sensor system of the invention, two dielectric layers are used. In one embodiment of the glucose sensor system, a first dielectric layer includes an aperture disposed above the working electrode defining an area of the working electrode. A second dielectric layer is formed into a capillary channel for a sample to be tested. The glucose sensor system of the invention also includes a top layer or lid material. In some embodiments, the lid material is a plastic material such as a polyester or polycarbonate plastic derivative. The lid material may be laminated with an adhesive to the top of a spacer dielectric layer to seal the capillary by providing a top wall to the capillary channel and complete the construction of the glucose sensor system of the invention.

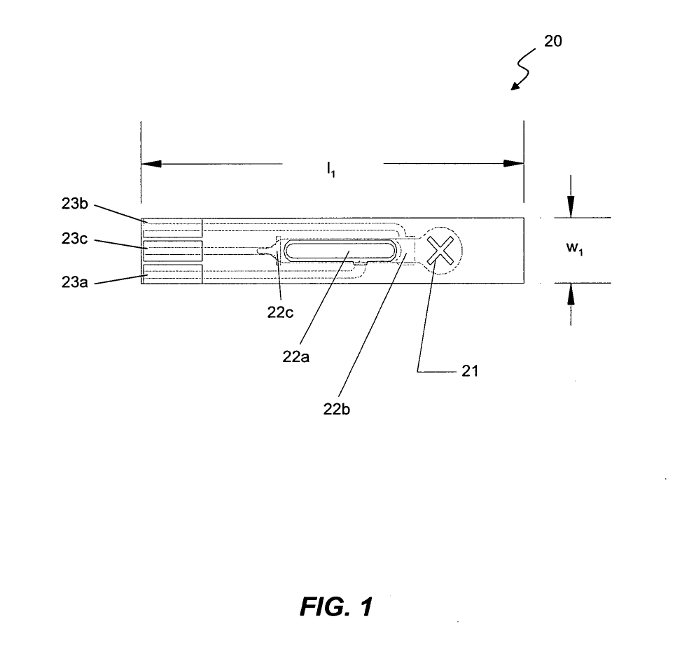

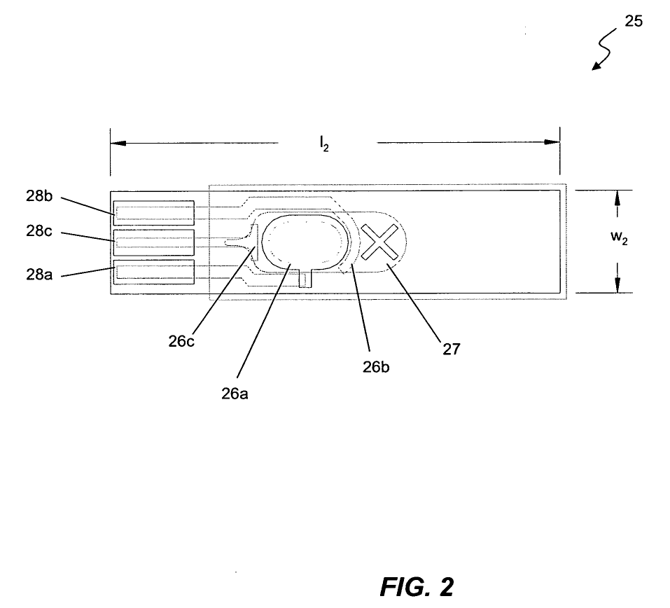

Some embodiments of the designs useful for the glucose sensor system of the invention are shown in FIG. 1 and FIG. 2. Referring to FIG. 1, a glucose sensor system 20 is rectangular having a length l₁ (e.g., 1.722 inch) and a width w₁ (e.g., 0.275 inch). The glucose sensor system 20 is planar including a sample port 21 accessible from a top surface, as shown. The system 20 also includes a working electrode 22a, a counter electrode 22b, and a fill-detect electrode 22c. The working electrode 22a is disposed between the sample port 21 and the fill-detect electrode 22c. The system 20 also includes three contacts 23a, 23b, 23c each in electrical communication with a respective one of the electrodes 22a, 22b, 22c.

Alternative embodiments of a rectangular glucose sensor systems, such as the system 5 shown in FIG. 2, can be formed having different geometries. The exemplary system 25 is also rectangular, having a different length l2 (e.g., 1.466 inch) and a width w2 (e.g., 0.340 inch) and a different configuration of the electrodes 26a, 26b, 26c, sample port 27, and contacts 28a, 28b, 28c.

Both designs 20, 25 have the same effective sample volume and electrode areas. The only substantial difference is in the overall shape of the sensor. Both designs use a capillary channel to feed the liquid across the electrodes. The large working electrode 22a, 26a (e.g., a platinum electrode) is relatively large and the reference electrode 22b, 26b (e.g., a Ag/AgCl electrode) is relatively smaller. The third electrode 22c, 26c that is used to detect the liquid as it reaches a vent area. The capillary height is set at least about 0.005 inch (125 microns).

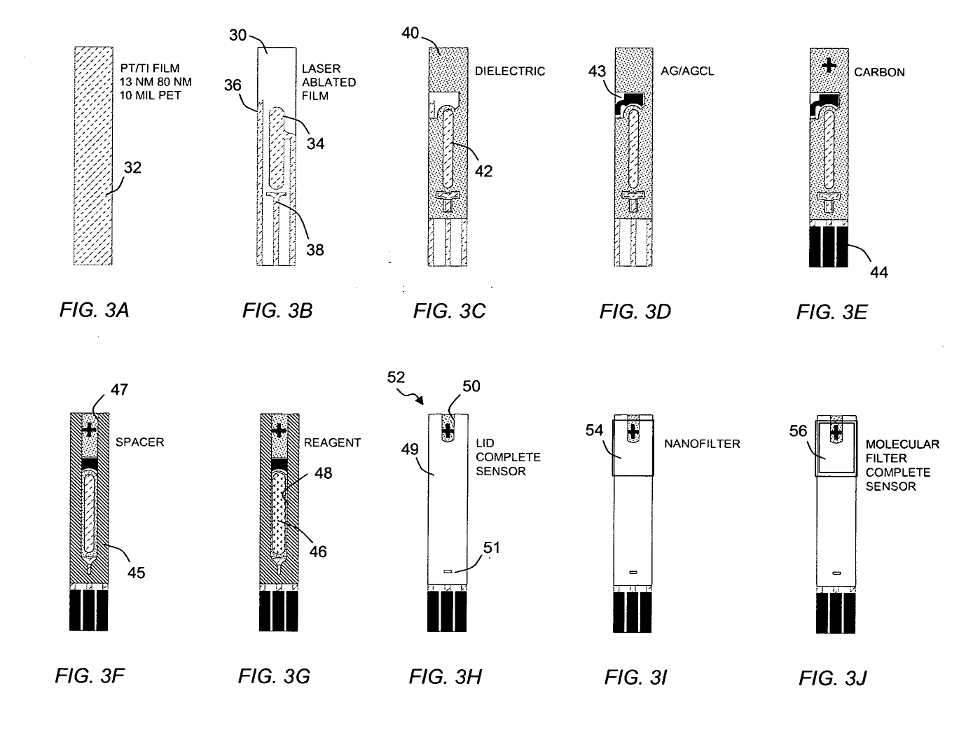

An embodiment of one design useful for the glucose sensor system of the invention is also shown in FIG. 3. In this embodiment, the sensing elements can be constructed using at least one of several technologies including laser ablation and screen printing. FIG. 3A through FIG. 3J show a step-wise build progression of a preferred embodiment of the glucose sensor system of the invention. A platinum/titanium film 32 on a plastic support 30 (FIG. 3B) is used as a base material. A key element is the working electrode 34 where the hydrogen peroxide is detected. This element 34 is shown as partially developed in FIG. 3B where this element (large, oval feature) 34 along with two other elements 36, 38 are formed by laser ablating selected regions of the thin, metal film 32 shown in FIG. 3A (e.g., about 13 nm Pt and about 80 nm Ti) from the underlying substrate 30 (e.g., about 0.010 inch PET). The working electrode 34 is further defined by a dielectric layer 40 (FIG. 3C) printed over the laser-ablated element 34. The dielectric layer 40 includes an aperture 42 at least partially aligned over the working electrode 34, defining a portion of the electrode 34 that will come in contact with the biological sample, e.g., saliva. Additional electrically conducting layers of Ag/AgCl 43 (FIG. 3D) and carbon 44 (FIG. 3E) are subsequently printed prior to depositing a second dielectric, or spacer layer 45 (FIG. 3F).

In the exemplary embodiment, the spacer layer 45 has a thickness of about 0.005 inch (5 mils) and is deposited prior to deposition of a reagent 46 as shown in FIG. 3G. The spacer layer 45 includes an elongated channel 47 forming side walls of a capillary channel and also partially defining the sample volume. The reagent 46, which includes glucose oxidase, is next added to the sensor during production by depositing an enzyme onto the exposed area of the working electrode 34 using an aqueous solution to create a measurement region 48 (FIG. 3G). During the assay, hydrogen peroxide formed over the measurement region 48 of the working electrode 34 is oxidized (i.e., loses electrons) at the working electrode surface to generate a detectable electrical signal in the system as shown below.

H₂O₂→O₂+2H⁺+2e⁻(+500 mV)

The final sensor element is a lid 49 (FIG. 3H). In one embodiment, the lid 49 includes a thin, hot-melt adhesive that is coated onto a 0.005″ PET substrate. This adhesive also has the property of being hydrophilic and therefore, it carries the burden of causing the sample to flow into the sensor. The lid 49 includes a first aperture 50 through which a sample gains access to a proximal end of the channel 47. The lid 49 also includes a second, generally smaller aperture at a distal end of the channel providing a vent 51 to the channel. The vent 51 can be formed by laser cutting.

During sensor fabrication the lid 49 can be laminated onto the sensor base using a heated roller. The sensor 52 is designed to fill with approximately 7 μL of sample. In one embodiment, the area of the working electrode 34 is less than about 20 mm². As such, the signal detection over the range of 50 μM to 1.5 mM glucose concentration in the sample requires that a fixed potential of +500 mV be applied and the resulting current be measured. In some embodiments, a small metal electrode near the vent hole 52 is a fill detect electrode 38 (FIG. 3B), while the Ag/AgCl near the sample entry point is the reference, or counter electrode 43 (FIG. 3D).

After the lid 49 is applied to the sensor 52, one or more additional materials 54, 56 (FIG. 31, FIG. 3J) can be adhered in the vicinity of the sample entry point 50 to treat or process the saliva sample prior to introduction into the sensor system. In one embodiment of the glucose sensor system of the invention, comprises a first material 56 that treats or processes the saliva sample as part of a sample purification. In some embodiments, this first treatment process is based on molecular absorption. Molecular absorption can be based on the use of discrete molecular size. This active molecular process constitutes the differential molecular separation of closely related molecular species based on the principle of selective absorption. To facilitate such at the molecular level in the case of glucose (e.g., about 180 Daltons), a variety of absorptive materials 54, 56 are available having controlled pore size to allow glucose to enter and pass unhindered through the absorptive matrix. This allows for final separation of glucose from salivary materials at the molecular level. Porous absorbents useful in the glucose sensor system of the invention are readily available with intra-particle pore sizes to allow entry of molecules around 300 MW (preferred) for glucose entry and internal surface areas up to 700 m²/gm. Such absorbents include, e.g., but are not limited to synthetic or natural Zeolite based materials, aluminum oxide micro spheres, synthetic ceramic micro spheres, hydrous alumina silicate micro spheres, additional natural clay absorbents, or activated carbon. Transfer of a portion of the sample across such materials is promoted by one or more of gravity and hydrophilic properties of the material.

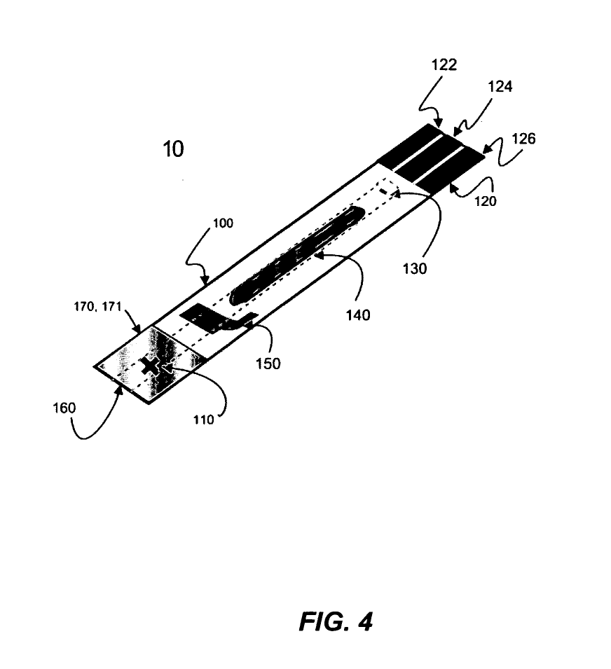

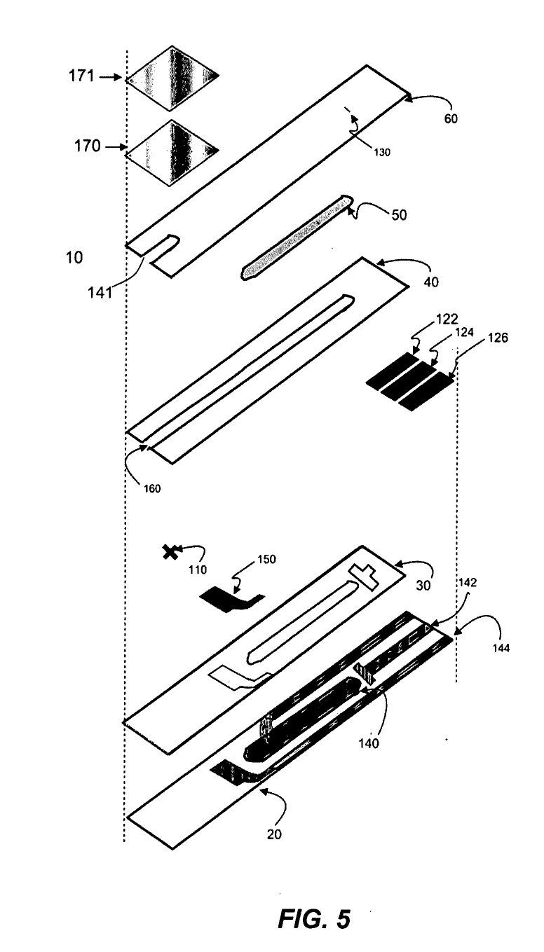

A preferred embodiment for the design of the glucose sensor system of the present invention is shown in FIG. 4 and FIG. 5. FIG. 4 shows the sensor 10 of the glucose sensor of the present invention. Sensor 10 has a laminated body 100, a fluid sampling port 110, an electrical contact end 120, and a vent opening 130. The fluid sampling port 110 is in fluid communication with a sample fluid channel 160 extending between the sampling port 110 and the vent opening 130. In addition, the fluid sampling port 110 is covered by a Nanofiltration material such as polycarbonate membrane 170 and a porous absorbent material such as a Zeolite membrane 171. The electrical contact end 120 has at least three direct conductive contacts 122, 124 and 126. Also shown are the working electrode 140 and the reference electrode 150. Each of the contacts 122, 124, 126 is in electrical communication with a respective one of the electrodes 140, 142, 150.

Referring to FIG. 5, the laminated body 100 is composed of a base insulating layer 20, a dielectric layer 30, a spacer layer 40, and a lid layer 60. All layers are made of a dielectric material, preferably plastic. Non-limiting examples of dielectric materials useful in the glucose sensor of the invention are PET, polyester, polycarbonate, PVC, polysulfone, acrylic and polystyrene. The base insulating layer 20 has conductive layer on which is delineated a first conductive conduit 140 (the working electrode), a second conductive conduit 142 (the fill electrode) and a third conductive conduit 144 (the reference electrode). In some embodiments, a pattern of the conductive layer is formed by laser ablating a thin metal film (e.g., 13 nm Platinum and 80 nm Titanium) from the underlying insulating layer 20. The second dielectric layer 30 has three cutouts for the reference 144, working 140 and fill detect electrodes 142. A silver/silver chloride (Ag/AgCl) layer 150 is printed over the part of the reference electrode 144 which comes in contact with the sample. Three carbon contacts, 122, 124, 126 are printed over the distal ends of the conductive conduits to maximize contact with measurement meter contact. Additionally a mark, such as a plus sign ‘+’ 110 is printed out of carbon material to indicate the position of the sample port 141. A spacer layer 40, approximately 0.005 inches thick is printed prior to reagent disposition. This layer forms the walls of the capillary channel 160 and partially defines the sample volume. Glucose oxidase 50 is deposited over that portion of the working electrode left exposed by the capillary channel 160. The final sensor element is the lid material 60. The lid material 60 is preferably hydrophilic substantially contributing to a capillary flow of a sample solution to the electrodes. The lid material 60 also contains a vent port 130. In some embodiments, the sample port 141 is covered by a nanofiltration material, such as polycarbonate membrane 170 and a porous absorbent material, such as a Zeolite membrane 171.

The carbon contacts, 122, 124, 126 provide contact with corresponding contacts of a separate measurement device, or meter (not shown) providing sensing circuitry. In some embodiments, an electrical potential is applied between two of the contacts 122, 126 providing a potential between the working electrode 140 and reference electrode 144. The voltage drop across these two electrodes with sample addition can then be used as a current measurement through the sensor circuit. For example, application of an external 500 mV source to the electrode contacts provides sufficient current for making a measurement. A current detector can be applied between the same contacts of the measurement device to detect an electrical current indicative of the electrochemical reaction. Results of the detected current level, along with other variables can be provided to a separate processor executing pre-programmed instructions for analyzing the sample to provide useful results, such as a glucose concentration of the sample.

Saliva Processing Feature of the Glucose Sensor System of the Invention.

Suitable samples for salivary glucose monitoring using the one-step devices 10 described herein comprise unstimulated or stimulated mixed whole saliva. The minimal sample volume that typically needs to be delivered to an electrochemical sensor strip is about 3 micro liters (μl). Most sensors work best with about 5 μl with no upper volume restraint. Any saliva collection device will need to reliably deliver a minimal volume of processed saliva (approx. 7 μl). Fluid may move through the sensor by any means deemed necessary. In one embodiment of the invention, delivery of the sample volume of processed saliva is via a passive process (i.e., capillary action and or gravity). However, in the case of membranes, saliva can be forced through the membrane (vertical flow) or along the membrane (horizontal flow) depending upon the need.

In one embodiment, the glucose sensor system of the invention comprises a membrane material which is useful to process or treat the saliva sample based on the principle of differential molecular nanofiltration. The nanopore membrane properties unique for saliva use include: nanopore size level of filtration; highly hydrophilic; non-clogging; thin; and able to withstand pressure or vacuum. As concerns active processes the recent advent of these membranes provides a technical means to selectively remove insoluble or soluble materials from samples in the range from 2 nanometers to several hundred million nanometers in a rapid fashion (e.g., less than about 30 sec).

Nanofiltration of a saliva sample with a 2 nm nanofilter results in a composition that passes the filter which contains soluble protein-like material below about 1,500 kDa; about 20 nm, and about 15,000 Daltons. Hydrophilic nanopore membranes useful for processing or treatment of saliva in the glucose sensor system of the invention are available commercially. For example, materials useful in the glucose sensor system of the invention include, but are not limited to, e.g., membranes with nanopores in the 2 to 200 nm size or above include ion track-etched polycarbonate membranes, inorganic aluminum oxide membranes, SPI-Pore Polycarbonate Membranes, commercially available from SPI Supplies of West Chester, Pa. and/or Steriltech ceramic disc membranes (comprised of alumina, zircocnia, or titania composites), commercially available from Steriltech of Omaha, Nebr. and/or any custom nanofabricated, uniform morphology, self-organized, anodic alumina nanodevice arrays constructed for thin film separation purposes. The transfer of saliva across such hydrophilic membrane material or nanoarrays is assisted by the hydrophilic nature of the sample channel itself. In one embodiment, of the glucose sensor system of the invention comprises a combination of materials useful for processing or treatment of saliva based on molecular absorption and molecular nanofiltration. Other methods related to glucose processing are described in International Patent Application Serial No. PCT/US2005/032466 filed on Sep. 12, 2005 under the Patent Cooperation Treaty, which claims priority to U.S. Provisional Patent Application Nos. 60/609,388 filed on Sep. 13, 2004, and 60/608,796 filed on Sep. 10, 2004, the contents of which are incorporated herein by reference in their entireties.

Methods of Use of the Glucose Sensor System of the Invention.

In another aspect, the invention provides a method determining glucose levels in a mammalian saliva sample using the glucose sensor system of the invention. In one embodiment, the invention the method comprises a processor wherein the processor correlates salivary carbohydrate levels in the sample with reference blood carbohydrate levels thereby calculating a range of probable carbohydrate levels based on the saliva sample carbohydrate levels and having an output for displaying information calculated by the processor. In one embodiment of the method of the invention, the processor correlates salivary carbohydrate levels of a subject/user of the glucose sensor system of the invention with historical carbohydrate levels or historical salivary carbohydrate levels of the subject/user of the device. In one embodiment of the method of the invention, the processor correlates salivary carbohydrate levels of a subject/user of glucose sensor system of the invention with historical medical or lifestyle information of the subject/user. In one embodiment of the method of the invention, the processor correlates carbohydrate levels of the subject/user of the glucose sensor system with blood glucose or blood hemoglobin al c values obtained by the subject/user by other or similar means. Alternatively or in addition, the processor correlates carbohydrate levels of the subject/user of the glucose sensor system of the invention with genetic information about the subject/user of the glucose sensor system of the invention. In one embodiment of the method of the invention, the output displays information indicating an appropriate therapeutic insulin dosage for the subject/user of the glucose sensor system of the invention.

A better understanding of the present invention and of its many advantages will be had from the following examples, which further describe the present invention and given by way of illustration. The examples that follow are not to be construed as limiting the scope of the invention in any manner. In light of the present disclosure, numerous embodiments within the scope of the claims will be apparent to those of ordinary skill in the art.

### EXAMPLES

Several types of sensors or sensor materials were employed in the studies described herein to demonstrate their utility for saliva glucose measurement. These included a most sensitive blood glucose sensor on the market and an embodiment of the glucose sensor system of the present invention.

### Example 1

### Blood Glucose Monitors Lack Sensitivity to Determine Salivary Glucose

For this study, the most sensitive commercially available blood glucose sensor on the market known by the applicants was employed. The EZ SMART blood glucose sensor, manufactured by Tyson BioResearch, Inc., Taiwan, ROC, has a published claimed sensitivity of 20 mg/dL. The sensor design for this product utilizes a carbon/carbon electrode on conventional base with a glucose oxidase/horse radish peroxidase enzyme channeling system for the amperometric electrochemical detection of glucose.

Glucose standards were prepared in phosphate buffered saline solution (PBS) at concentrations between 0.0 and 6.0 mg/dL. Samples were measured in triplicate using the commercial blood sensor strip. Current in microamps (μA) was measured using a laboratory potentiometer to maximize efficiency. The control instrument was the YSI 2700 laboratory based reference instrument, which measures glucose analytically by electrochemical means. Results are summarized below in Table 1.

TABLE 1
Comparison of EZ SMART and YSI Glucose Monitor Sensitivity

| Glucose | EZ SMART | |
|---|---|---|
| mg/dL | μA | YSI 2700 μA |
| 0.238 | 0.01331 | 0.01 |
| 1.56 | 0.01603 | 0.07 |
| 3.21 | 0.01777 | 0.15 |
| 4.62 | 0.01964 | 0.21 |
| 6.24 | 0.02146 | 0.28 |

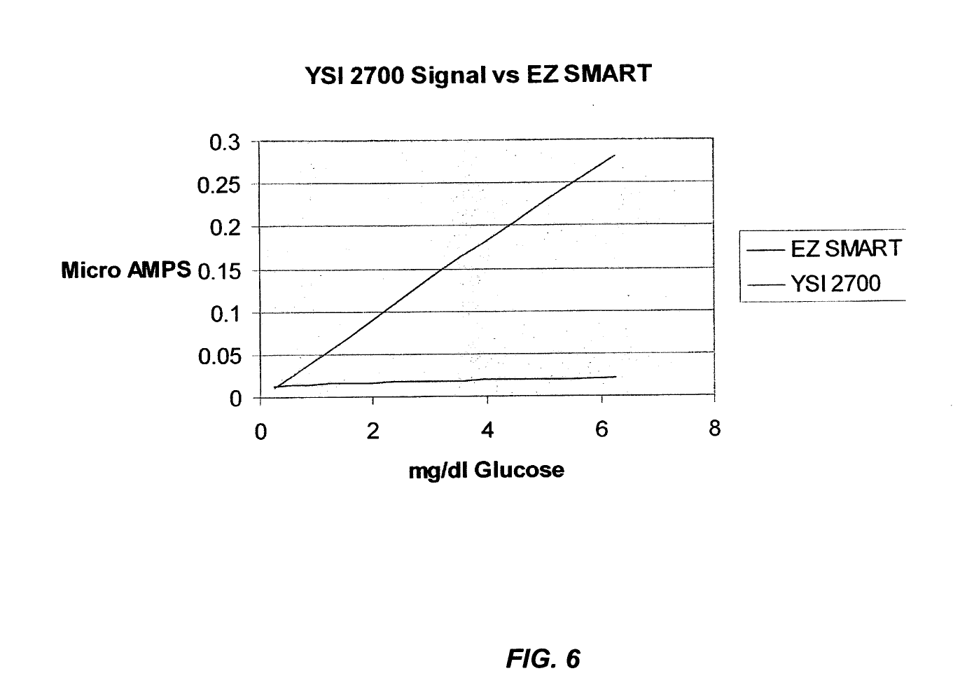

FIG. 6 also shows a comparison of the sensitivity of YSI 2700 and EZ SMART glucose monitors. Table 1 and FIG. 6 demonstrate the inability of the commercial sensor EZ SMART to measure glucose in the dynamic range required for salivary monitoring use. Saliva glucose levels range from 0.0 to 5 mg/dL. Both noise and lack of signal contributed to poor performance in this range. The YSI 2700, on the other hand was able to measure glucose at these low levels as the analytical reference method used in the research laboratory. The YSI 2700 is a research tool (not used clinically), used in this study as a control to confirm that standard solutions prepared contained glucose and that it was measurable.

### Example 2

### Selection of Material Useful in the Glucose Sensor System of the Invention

Studies to demonstrate the utility of materials included the identification and selection of a suitable electrode materials for sensitivity to H₂O₂ measurement followed by the demonstration of a stepwise dose-response curve in the operating range required for saliva using electrode film material. For the first analysis, a variety of electrode materials were used for testing including gold or palladium sputtered films, carbon printed films, and gold or platinum pellet material. The original parts sent for testing were printed on a 0.005 inch Melinex polyester material, using five separate thick film inks. The leads, coming from the connector end to just before the working areas were printed using a silver loaded thick film ink. The reference electrode was screened with a silver/silver chloride material, while the counter electrode was comprised of a carbon loaded material. The working electrode was printed using a modified carbon thick film ink. The final printed layer was a UV cured dielectric, used to coat the leads and define the working area of the sensor. Individual parts were then removed from the array using a laser cutting system, production parts were cut using a hand tool, and pins crimped to the connector end for attaching to testing equipment.

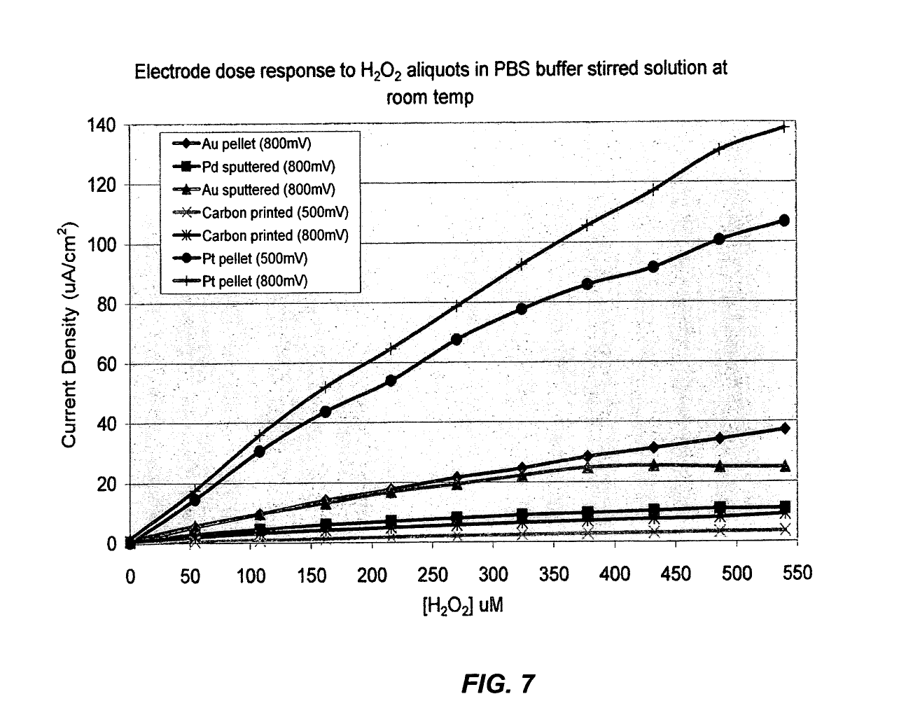

Parts for original review did not contain spacer printing or the lid to complete the capillary channel. After attachment to testing equipment, electrode dose responses to H₂O₂ were measured in response to aliquots of H₂O₂ to stirred 100 mM PBS. Current density in microamps/cm² was measured using a conventional laboratory potentiometer. The results of this study are summarized in FIG. 7.

FIG. 7 shows that no dose response curve was observed for conventional carbon printed electrodes. This finding was consistent with the results of studies presented in Example 1, which utilized a commercial blood sensor using the same carbon printed electrode material. Palladium sputter deposited metallized film gave a marginal dose response. Gold pellet or sputter deposited material gave a slightly better dose response, but platinum materials clearly gave the best dose response curve exhibiting a signal and noise suitable for detection of salivary glucose. The dose response noted with platinum material mirrored the responses observed with the YSI 2700 analytical reference instrument in Example 1.

### Example 3

### Effect of Disposition Method on Amperometric Signal

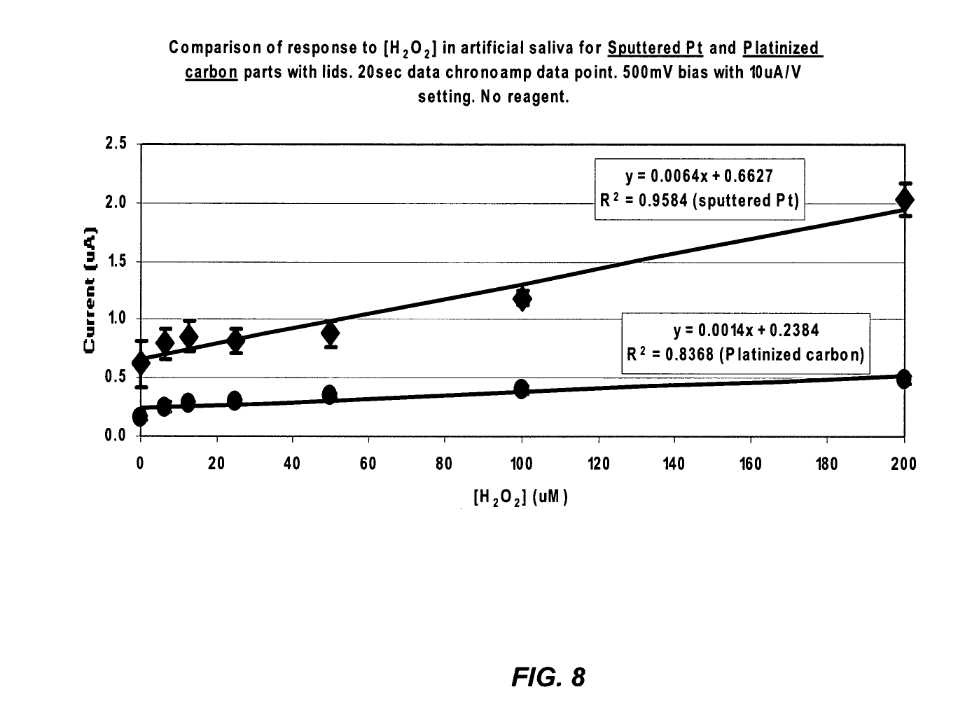

To demonstrate a dose-response curve in the operating range required for saliva a simple platinized/printed carbon and a sputtered platinum electrode films were constructed as described in Example 2. The platinized/carbon working electrode film and a sputtered platinum were compared to a smaller reference electrode (Ag/AgCl) for measurement of H₂O₂. Results are summarized in FIG. 8, which shows the stepwise dose response of the electrodes to H₂O₂ a 0 to 500 μM concentration range. Noise was negligible and signal response showed both reasonable amplitude and stepwise response to H₂O₂ over the concentration range required for saliva glucose monitoring.

### Example 4

### Effect of Glucose Oxidase Concentration of Amperometric Response

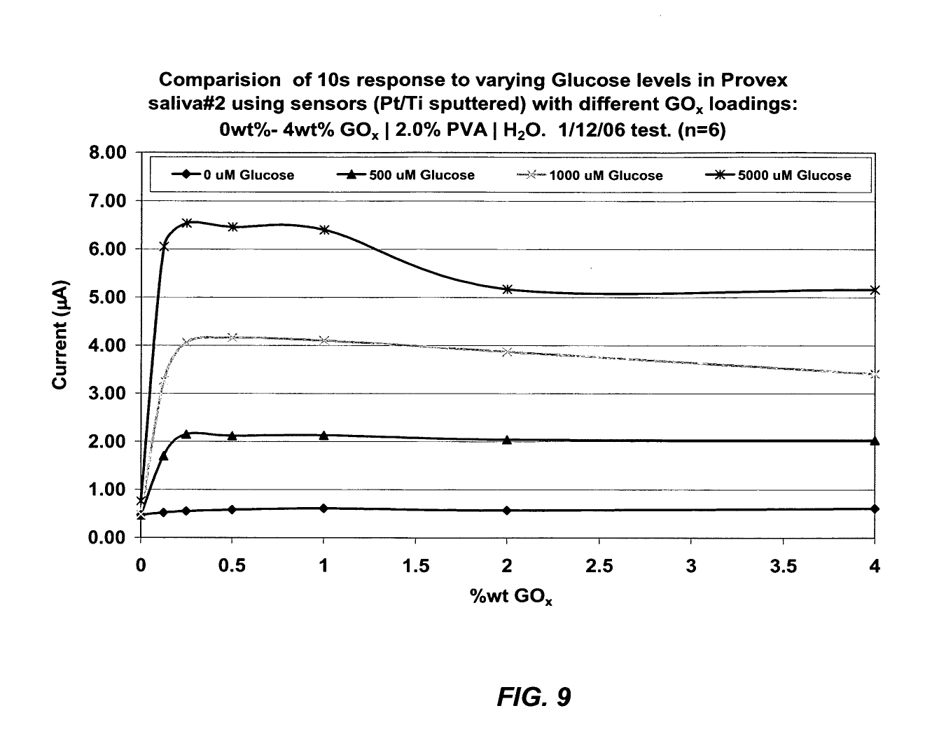

To determine the concentration of glucose oxidase (GOx) required for maximum amperometric response the effect of varied GOx levels was measured over a range of glucose concentrations (0-5000 μM). The results are shown in FIG. 9. As shown in the figure, 1 wt % GOx concentration yielded a maximum amperometric response over the entire glucose concentration range tested and allowed for inactivation or instability of the enzyme over time since the lower concentrations give equivalent response between 1 wt % and 0.25 wt % GOx. Accordingly, 1 wt % GOx identified as at least one concentration of the enzyme for use in the glucose sensor system of the invention.

### Example 5

### Characterization of a Glucose Sensor System of the Invention

Studies were conducted to determine the sensitivity, resolution and signal to noise of two representative lots of the glucose sensor system of the invention of a design as shown in FIG. 2 with 1 wt % GOx with Pt/Ti 130 A using two formulations: no buffer or phosphate buffer. The results of each lot are summarized in Table 2 and Table 3 shown below. The platinum sensor described in FIG. 3, without the nanofilter or the molecular filtration layers was used to measure the current of the samples.

TABLE 2
Sensitivity, resolution and signal to noise for 1 wt % GOx (No Buffer)

| Glucose | Average | Std. | | | |
|---|---|---|---|---|---|
| level | Current | Dev. | Avg. + 1 SD | Avg. − 1 SD | Coefficient of |
| (μM) | (μA) | (SD) | (μA) | (μA) | Variation (%) |
| 0 | 2.03 | 0.082 | 2.112 | 1.948 | 4.02 |
| 25 | 2.090 | 0.092 | 2.182 | 1.998 | 4.41 |
| 50 | 2.216 | 0.127 | 2.343 | 2.089 | 5.72 |
| 200 | 2.868 | 0.108 | 2.978 | 2.762 | 3.78 |
| 500 | 3.948 | 0.179 | 4.129 | 3.771 | 4.54 |
| 1000 | 6.583 | 0.628 | 7.208 | 5.952 | 9.54 |
| 2000 | 9.092 | 0.568 | 9.658 | 8.522 | 6.24 |
| 5000 | 9.815 | 0.479 | 10.299 | 9.341 | 4.88 |

TABLE 3
Sensitivity, resolution and signal to noise for 1 wt % GOx (Phosphate
Buffer)

| Glucose | Average | Std. | | | |
|---|---|---|---|---|---|
| level | Current | Dev. | Avg. + 1 SD | Avg. − 1 SD | Coefficient of |
| (μM) | (μA) | (SD) | (μA) | (μA) | Variation (%) |
| 0 | 1.48 | 0.131 | 1.611 | 1.349 | 8.89 |
| 25 | 1.603 | 0.148 | 1.748 | 1.452 | 9.25 |
| 50 | 1.778 | 0.064 | 1.842 | 1.714 | 3.57 |
| 200 | 2.744 | 0.095 | 2.235 | 2.045 | 3.47 |
| 500 | 4.616 | 0.385 | 5.005 | 4.235 | 8.35 |
| 1000 | 8.780 | 0.501 | 9.281 | 8.279 | 5.71 |
| 2000 | 12.320 | 0.844 | 13.164 | 11.476 | 6.85 |
| 5000 | 14.030 | 0.688 | 14.718 | 13.342 | 4.9 |

The area of each electrode was 19.322 mm². They were tested on BAS electrochemical analyzer with n=10. Various glucose levels were made in saliva #2. Seven microliters of test solution was used per sensor.

The detection limit was obtained using the classical approach where the minimum distinguishable signal is equal to the mean signal plus three standard deviations of the blank. The limit of detection determined using this method was 55.1 μM glucose and 53.8 μM glucose for the formulation without buffer (Table 2) and the formulation with phosphate buffer (Table 3), respectively. The slope [nA/μM]/mm² Pt electrode was determined as about 0.233 [nA/μM]/mm² Pt electrode and 0.378 [nA/μM]/mm² Pt electrode for the formulation without buffer (Table 2) and the formulation with phosphate buffer (Table 3), respectively. The electrodes showed good response down to about the 25 to 50 μM glucose concentration range. The mean current (μA) with ± one standard deviation is shown in the table. Some overlap between the 25 μM glucose and 50 μM glucose test range was observed. Thus, as a first measure the sensor would likely have a resolution greater than 25 μM glucose.

### Example 6

### Effect of Electrode Surface Area on the Responsiveness of an Embodiment of the Glucose Sensor System of the Invention

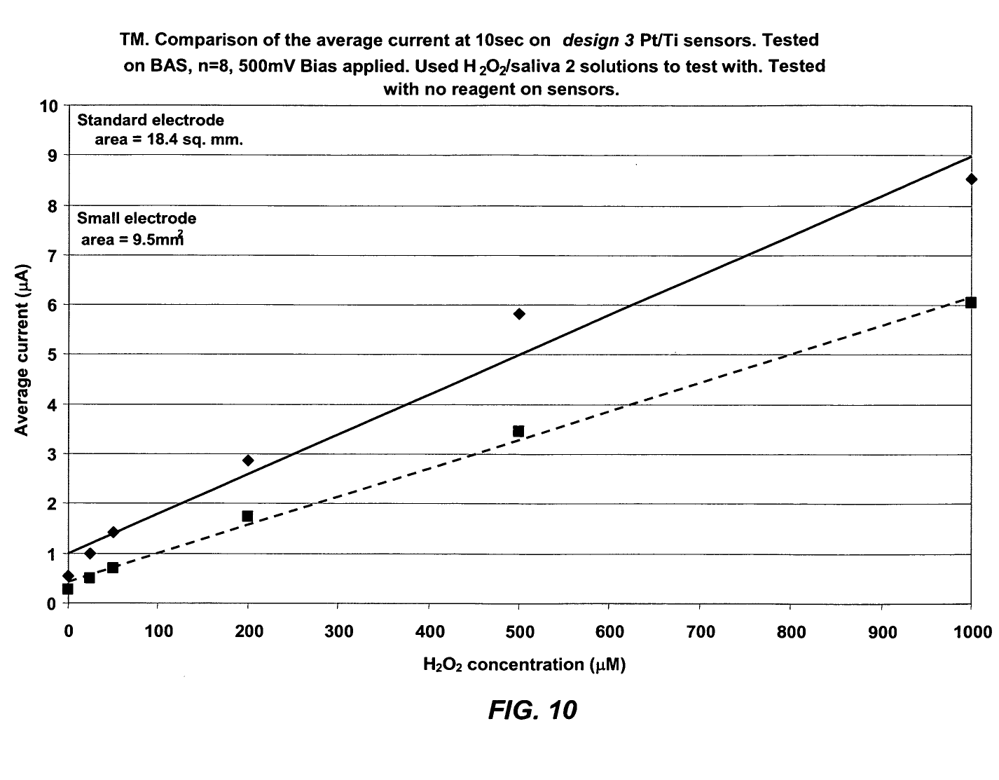

Studies were conducted to examine the effect of varying surface area of the working electrode the responsiveness of a glucose sensor system as described in FIG. 4. As shown in FIG. 10, the response observed over a concentration range of H₂O₂ concentrations (e.g., about 0 to about 1000 μM) is directly proportional to the surface area of the working electrode. The surface area of sensor in either case is large and much larger than blood sensors to afford saliva sensitivity and high signal production.

Equivalents.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains.

What is claimed is:

1. An apparatus for detecting a concentration of glucose in a biological sample other than blood, comprising:
a support member;
an electrode disposed on the support member having an exposed surface area;
a sample region comprising a sample port upon which the biological sample is applied;
a lumen having a proximal end in fluid communication with the sample region; and
a measurement zone in fluid communication with a distal end of the lumen, the measurement zone having an enzymatic catalyst, the measurement zone providing a minimum sensitivity of at least about 50 micromolar glucose concentration and a noise level of less than about 0.5 nA/μM/mm².

2. The apparatus of claim 1, wherein the biological sample is saliva.

3. The apparatus of claim 1, wherein the electrode is a multi-layer electrode.

4. The apparatus of claim 3, wherein the multi-layer electrode comprises a platinized carbon electrode layer deposited on a plastic film layer.

5. The apparatus of claim 3, wherein the multi-layer electrode includes at least one metal selected from the group consisting of: gold; palladium; carbonized platinum; and platinum.

6. The apparatus of claim 3, wherein the multi-layer electrode includes a platinum layer over a titanium layer.

7. The apparatus of claim 6, wherein the plastic film comprises at least one of polyvinylidene difluoride (PVDP) and polyethylene terephthalate (PET) as a solid phase.

8. The apparatus of claim 1, wherein the enzymatic catalyst is glucose oxidase.

9. The apparatus of claim 8, wherein the glucose oxidase is present at a concentration of at least about 1 weight percent.

10. The apparatus of claim 1, wherein the support comprises one or more conductive coatings for forming at least three electrodes including a working electrode, a fill electrode, and a reference electrode.

11. The apparatus of claim 10 wherein the conductive coatings comprise at least one metal or metal salt selected from the group consisting of: carbon, gold, palladium platinum, titanium, silver, and silver chloride.

12. The apparatus of claim 10, wherein the support further comprises one or more layers selected from the group consisting of: a dielectric layer, a spacer layer and a lid layer.

13. The apparatus of claim 12, wherein the one or more layers are formed from a plastic selected from the group consisting of: polyethylene terephthalate (PET), polyester, polycarbonate, polyvinyl chloride (PVC), polysulfone, acrylic and polystyrene.

14. The apparatus of claim 13, further comprising a filter for filtering the biological sample.

15. The apparatus of claim 14, wherein the filter comprises a nanofiltration material and a porous absorbent material.

16. The apparatus of claim 15, wherein the nanofiltration material is a hydrophilic nanopore membrane selected from the group consisting of: ion track-etched polycarbonate nanomembranes; inorganic aluminum oxide nanomembranes; polyester nanomembranes; and composite ceramic nanopore membranes.

17. The apparatus of claim 15, wherein the porous absorbent material is selected from the group consisting of: a zeolite; aluminum oxide microspheres; ceramic microspheres; hydrous alumina silicate microspheres; alumina dessicant microbeads; attapulgus clay beaded silica gel dessicants; natural clay absorbents; natural clay adsorbents; activated carbon; and combinations thereof.

18. The apparatus of claim 1, wherein the exposed surface area is at least about 10 mm².

19. The apparatus of claim 1, wherein the measurement zone provides a sensitivity from about 0 milligrams per deciliter glucose concentration to about 30 milligrams per deciliter glucose concentration.

20. An apparatus for processing a mammalian saliva sample comprising:
a saliva sample port for receiving the saliva sample; and
a filter in fluid communication with the sample port, the filter including:
a nanofiltration material configured to remove high molecular weight contaminants from the saliva sample; and
a porous absorbent material configured to absorb at least a portion of the saliva sample,
wherein the saliva sample is filtered by the nanofiltration material and the absorbent material.

21. The apparatus of claim 20, wherein the nanofiltration material comprises a hydrophilic nanopore membrane selected from the group consisting of: ion track-etched polycarbonate nanomembranes; inorganic aluminum oxide nanomembranes; polyester nanomembranes; composite ceramic nanopore membranes; and combinations thereof.

22. The apparatus of claim 20, wherein the porous absorbent material has an internal surface area greater than about 400 M²/gram.

23. The apparatus of claim 20, wherein the porous absorbent material is selected from the group consisting of: a zeolite; aluminum oxide microspheres; ceramic microspheres; hydrous alumina silicate microspheres; alumina dessicant microbeads; attapulgus clay beaded silica gel dessicants; natural clay absorbents; natural clay adsorbents; activated carbon; and combinations thereof.

24. The apparatus of claim 20, further comprising a sensor in communication with the saliva sample absorbed into the matrix, the sensor detecting glucose levels in the processed saliva sample.

25. The apparatus of claim 24, wherein the sensor is an electrochemical sensor configured for determining a concentration of an analyte via an electrochemical oxidation and reduction reaction.

26. The apparatus of claim 25, wherein the electrochemical sensor comprises:
a rigid support member;
a metallic electrode disposed on the rigid support member having a surface area of at least about 10 mm²;
a sample region comprising a sample port upon which the biological sample is applied;
a lumen having a proximal end in fluid communication with the sample region; and
a measurement zone in fluid communication with a distal end of the lumen, the measurement zone having an enzymatic catalyst, the measurement zone providing a minimum sensitivity of at least about 50 micromolar glucose concentration and a noise level of less than about 0.5 nA/μM/mm².

27. A method for determining glucose levels in a mammalian saliva sample comprising:
receiving the saliva sample at a sample port;
transporting at least a portion of the received saliva sample from the sample port to a measurement zone;
combining the transported saliva sample with an enzymatic catalyst within the measurement zone;
measuring a glucose level of the saliva sample with a minimum sensitivity of at least about 50 micromolar glucose concentration and a noise level of less than about 0.5 nA/μM/mm².

28. The method of claim 27, wherein the enzymatic catalyst is glucose oxidase.

29. The method of claim 28, wherein the glucose oxidase is present at a concentration of at least about 1 weight percent.

30. The method of claim 27, further comprising filtering the saliva sample using at least one of a nanofiltration material and a porous absorbent material.

31. The method of claim 27, further comprising filtering the saliva sample using a nanofiltration material and a porous absorbent material.

32. The method of claim 31, wherein the nanofiltration material is a hydrophilic nanopore membrane selected from the group consisting of: ion track-etched polycarbonate nanomembranes; inorganic aluminum oxide nanomembranes; polyester nanomembranes; and composite ceramic nanopore membranes.

33. The method of claim 31, wherein the porous absorbent material is selected from the group consisting of: a zeolite; aluminum oxide microspheres; ceramic microspheres; hydrous alumina silicate microspheres; alumina dessicant microbeads; attapulgus clay beaded silica gel dessicants; natural clay absorbents; natural clay adsorbents; activated carbon; and combinations thereof.

34. The method of claim 27, wherein the measurement zone provides a sensitivity from about 0 milligrams per deciliter glucose concentration to about 30 milligrams per deciliter glucose concentration.
